(19) United States
(12) Patent Application Publication
(10) Pub. No.: US 2025/0299793 A1
(43) Pub. Date: Sep. 25, 2025

(54) MEDICATION MANAGEMENT SYSTEM, METHOD, AND APPARATUS

(71) Applicant: TERUMO KABUSHIKI KAISHA, Tokyo (JP)

(72) Inventors: Masaru IKEGAMI, Fujisawa Kanagawa (JP); Yoshihito MACHIDA, Sagamihara Kanagawa (JP); Tomoko UEMURA, Hadano Kanagawa (JP); Megumi MIZUNO, Hadano Kanagawa (JP)

(21) Appl. No.: 19/224,922

(22) Filed: Jun. 2, 2025

Related U.S. Application Data
(63) Continuation of application No. PCT/JP2023/043556, filed on Dec. 6, 2023.

(30) Foreign Application Priority Data
Dec. 6, 2022 (JP) 2022-195025

(51) Int. Cl. G16H 20/10

(52) U.S. Cl. CPC G16H 20/10

(57) ABSTRACT

A medication management system includes a display device, a memory, and a processor configured to acquire patient information about a patient, a first test value of a laboratory test performed on the patient, and medicine information indicating a medicine to be administered, execute a call to a machine learning model with the patient information, the first test value, and the medicine information to determine a second test value expected at a predetermined time after the medicine is administered, the model trained with test values obtained before and after administration of the medicine to different patients, determine a recommended dose or a preferred range of doses for the medicine based on the second test value, generate a graph showing a relationship between doses of the medicine and test values, and display the graph and the recommended dose or the preferred range on the graph.

### CROSS-REFERENCE TO RELATED APPLICATION

This application is a continuation of International Patent Application No. PCT/JP2023/043556 filed Dec. 6, 2023, which is based upon and claims the benefit of priority from Japanese Patent Application No. 2022-195025, filed Dec. 6, 2022, the entire contents of which are incorporated herein by reference.

### BACKGROUND

### Technical Field

Embodiments described herein relate to a medication management system, method, and apparatus.

### Background Art

Heparin, used as an anticoagulant in intensive care units, needs to be carefully dosed, but guideline-recommended dosing based on patient body weight is not optimal in real clinical settings. Moreover, the pathophysiology of coagulation is complex and not yet fully understood. Therefore, there is growing interest in applying machine learning prediction models to support clinical judgment based on many parameters.

For example, there is a publication that describes a prediction model for activated partial thromboplastin time (APTT) using data from a large number of patients. Such a model predicts APTT several hours after the start of administration of heparin to calculate an appropriate dose of heparin.

Medical workers adjust the heparin dose using test parameters such as APTT and activated clotting time (ACT), but some medical facilities and clinical departments measure only a subset of these test parameters. Under such circumstances, a predictive system that uses test parameters such as APTT as explanatory variables may be less versatile, because it cannot be applied to patients whose data are not complete.

### SUMMARY

Embodiments of this disclosure provide a medication management system, method, and apparatus that can suitably predict an influence of administration of a medicine on a patient or a preferable dose of the medicine.

In one aspect, a medication management system for managing medication for a patient, comprises: a display device; a memory that stores a program; and a processor configured to execute the program to: acquire patient information about the patient, a first test value of a laboratory test that was performed on the patient, and medicine information indicating a dose of a medicine to be administered to the patient, input the patient information, the first test value, and the medicine information into a machine learning model to output a second test value expected at a predetermined time after the medicine is administered, the machine learning model having been trained with test values obtained before and after administration of the medicine to different patients, determine a recommended dose or a preferred range of doses for the medicine based on the second test value, generate data of a graph showing a relationship between doses of the medicine and test values, and control the display device to display the graph and the recommended dose or the preferred range on the graph.

In one aspect, it is possible to suitably predict an influence of administration of a medicine on a patient or a preferable dose of the medicine.

### BRIEF DESCRIPTION OF DRAWINGS

FIG. 1 is an explanatory diagram illustrating a configuration example of an information processing system.

FIG. 2 is a block diagram illustrating a configuration example of a server.

FIG. 3 is a block diagram illustrating a configuration example of a terminal.

FIG. 4 is an explanatory diagram illustrating an outline of a first embodiment.

FIG. 5 is an explanatory diagram illustrating a display example of a prediction result.

FIG. 6 is a flowchart illustrating processing for generating a machine learning model.

FIG. 7 is a flowchart illustrating prediction processing using the learning model.

FIG. 8 is a flowchart illustrating processing for generating a machine learning model according to a second embodiment.

FIG. 9 is a flowchart illustrating prediction processing using the learning model according to the second embodiment.

FIG. 10 is an explanatory diagram illustrating an outline of a third embodiment.

FIG. 11 is a flowchart illustrating processing for generating a machine learning model according to the third embodiment.

FIG. 12 is a flowchart illustrating prediction processing using the learning model according to the third embodiment.

### DETAILED DESCRIPTION

Hereinbelow, embodiments will be described in detail with reference to the drawings illustrating embodiments thereof.

### First Embodiment

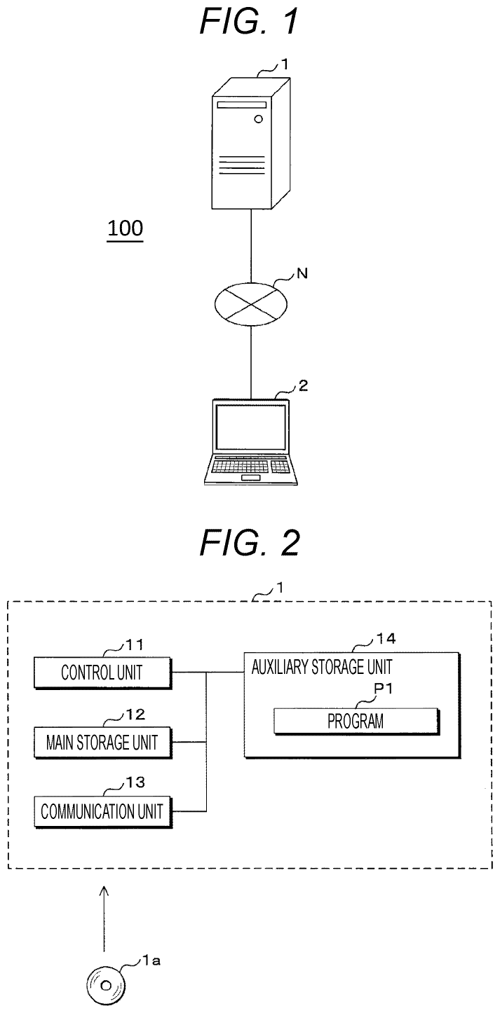

FIG. 1 is an explanatory diagram illustrating a configuration example of an information processing system 100. In the present embodiment, the information processing system 100 is a medication management system that predicts an influence of a medicine on a patient by administration of a medicine and/or a dose of the medicine to be administered using a machine learning model. The information processing system 100 includes a server 1 and a terminal 2. These devices are interconnected to communicate with each other via a network N, such as the Internet.

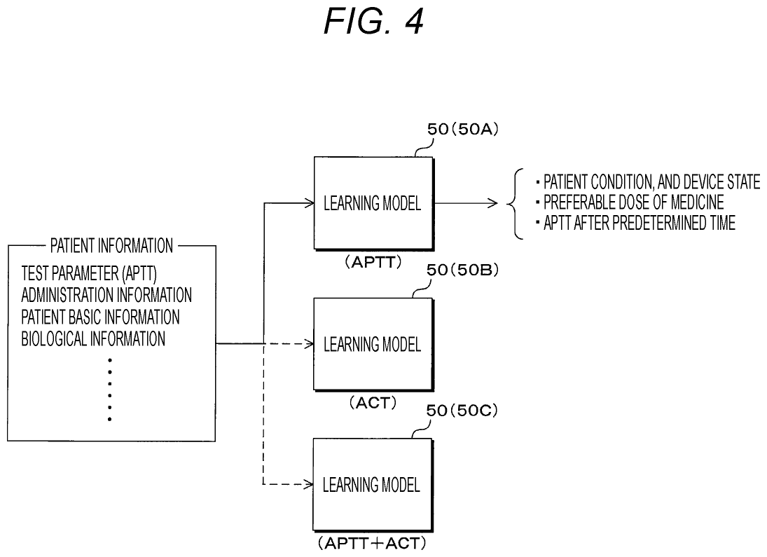

The server 1 is a medication management apparatus that can perform various types of information processing and transmit and receive information. The computer corresponding to the server 1 may be a personal computer or the like. As described below, the server 1 generates a machine learning model 50 (see FIG. 4) that outputs information regarding an influence of a medicine to be administered to a patient (possibility of occurrence of a complication or the like) and/or a dose of the medicine to be administered in a case where patient information including a test parameter (APTT or the like) of the patient is input by machine learning using predetermined training data. In particular, in the present embodiment, the server 1 generates a plurality of learning models 50 that have been trained using different pieces of training data including the test parameters of different types, so that an influence of a medicine and the like can be predicted in a common system even in a case where different test items (or parameters) are used depending on the medical institution. Note that, in the present application, the information regarding a dose of a medicine includes not only a dose of a medicine but also a range of a dose of a medicine (for example, a dose of heparin is in the range from 1000 U to 1500 U).

The terminal 2 is a terminal device used by a medical worker, and is, for example, a personal computer, a tablet terminal, and a smartphone. For example, data of the learning model 50 generated by the server 1 is installed in the terminal 2, and the terminal 2 predicts and outputs an influence of a medicine or the like as patient information is input into the learning model 50.

Note that, in the present embodiment, the description will be given assuming that the server 1 generates the learning model 50 and the terminal 2 performs prediction based on the learning model 50, but the two pieces of processing may be executed by a single computer.

FIG. 2 is a block diagram illustrating a configuration example of the server 1. The server 1 includes a control unit 11, a main storage unit 12, a communication unit 13, and an auxiliary storage unit 14.

The control unit 11 includes one or a plurality of arithmetic processing units such as a central processing unit (CPU), a micro-processing unit (MPU), and a graphics processing unit (GPU) and executes various types of information processing, control processing, and the like by loading and executing a program P1 stored in the auxiliary storage unit 14. The main storage unit 12 is a memory such as a static random access memory (SRAM), a dynamic random access memory (DRAM), and a flash memory and temporarily stores data necessary for the control unit 11 to execute arithmetic processing. The communication unit 13 is a network interface circuit that performs processing related to communication and transmits and receives information to and from the outside. The auxiliary storage unit 14 is a non-volatile storage device such as a high-capacity memory and a hard disk, and stores the program P1 necessary for the control unit 11 to perform processing and other data.

Note that the auxiliary storage unit 14 may be an external storage device connected to the server 1. In addition, the server 1 may be a multi-computer that includes a plurality of computers or may be a virtual machine implemented by software in a virtual manner.

In addition, in the present embodiment, the configuration of the server 1 is not limited to the above one, and the server 1 may include, for example, an input unit that receives an operation input and a display unit that displays an image. Moreover, the server 1 may include a reading unit that performs reading operations on a portable storage medium 1a, such as a compact disk (CD)-ROM and a digital versatile disc (DVD)-ROM, and read the program P1 from the portable storage medium la and then execute the program.

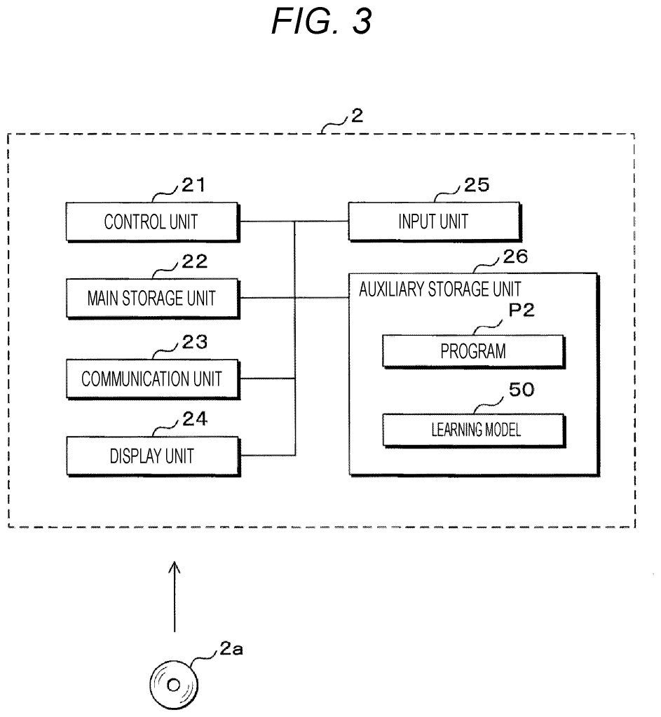

FIG. 3 is a block diagram illustrating a configuration example of the terminal 2. The terminal 2 includes a control unit 21, a main storage unit 22, a communication unit 23, a display unit 24, an input unit 25, and an auxiliary storage unit 26.

The control unit 21 includes one or a plurality of processors such as a CPU and executes various types of information processing by reading and executing a program P2 stored in the auxiliary storage unit 26. The main storage unit 22 is a memory such as a RAM and temporarily stores data necessary for the control unit 21 to execute arithmetic processing. The communication unit 23 is a network interface circuit that performs processing related to communication and transmits and receives information to and from the outside. The display unit 24 is a display such as a liquid crystal display, and displays an image. The input unit 25 is an operation interface such as a touch panel, and receives an operation input from a user.

The auxiliary storage unit 26 is a non-volatile storage device such as a hard disk, and stores the program P2 necessary for the control unit 21 to perform processing and other data. In addition, the auxiliary storage unit 26 stores the learning model 50. The learning model 50 is a machine learning model that has been trained using predetermined training data so as to output information regarding an influence of a medicine and/or a dose of the medicine to be administered in a case where patient information including a test parameter of the patient and administration information of the medicine is input.

Note that the terminal 2 may include a reading unit that performs reading operations on a portable storage medium 2a, such as a CD-ROM, and read the program P2 from the portable storage medium 2a and then execute the program.

FIG. 4 is an explanatory diagram illustrating an outline of the first embodiment. FIG. 4 illustrates a state in which patient information including test parameters of a patient is selectively input into any one of the plurality of different learning models 50 (50A, 50B, and 50C) according to the types of the test parameters to output an influence of a medicine (e.g., a condition of patient and the like) and/or a preferable dose of the medicine. With reference to FIG. 4, an outline of the present embodiment will be described below.

The learning model 50 is a machine learning model that outputs information regarding an influence of a medicine and/or a dose of the medicine to be administered in a case where patient information including a test parameter (e.g., APTT or the like) of a patient is input, and is, for example, a neural network trained using deep learning.

Note that the learning model 50 is not limited to a neural network and may be a computer model which is based on, for example, a decision tree, a support vector machine (SVM), or another learning algorithm.

The learning model 50 according to the present embodiment uses the following patient information as explanatory variables (or input values).

(1) Patient test parameters (APTT, ACT, platelet count, red blood cell count, white blood cell count, hematocrit, Na, K, Ca, Cl, creatinine, BUN, PO2, PCO2, pH, and the like) and test date and time, (2) Medicine administration information (medicine name (drug name such as heparin, infusion solution, and catecholamine), administration route, administration rate, administration amount, scheduled administration start date and time, and the like), (3) Patient basic information (age, sex, height, weight, blood type, and the like), (4) Biological information (blood pressure (systolic, diastolic, and mean), SP02, body temperature, respiratory rate, pulse rate, level of consciousness, and the like), (5) Information regarding connected device (Extracorporeal Membrane Oxygenation (ECMO)) (start date and time, end date and time, type of connected device, cannula size, centrifugal pump rotation speed, blood flow rate, gas flow rate, and the like), (6) Surgical information (surgical method, type of surgery, bleeding amount, infusion amount, and the like), and (7) Treatment information (Presence or absence of dialysis, presence or absence of ventilator, and the like)

The learning model 50 receives as input patient information and outputs at least one of the following output values. Note that the patient information may be input into the learning model by a medical worker at the time of prediction, or the patient information already input and stored in the database may automatically be input into the learning model.

(i) Condition of patient and/or state of connected device after predetermined time (possibility of complication, possibility of thrombogenicity in ECMO circuit, and the like) (ii) Numerical value or numerical range of preferable dose of medicine (iii) Value of laboratory test parameter (APTT, ACT, or both APTT and ACT) after predetermined time

The server 1 generates or optimizes the learning model 50 using training data in which a correct output value is associated with patient information for training. Here, in the present embodiment, the server 1 uses a plurality of pieces of training data having different test parameters (APTT, ACT, and both APTT and ACT) to generate the plurality of learning models 50A, 50B, and 50C having input therein different types of test parameters.

FIG. 4 illustrates a state in which three types of learning models 50A, 50B, and 50C are prepared. The input parameter of the learning model 50A is APTT, the input parameter of the learning model 50B is ACT, and the input parameters of the learning model 50C are APTT and ACT. The server 1 generates the learning model 50A in which APTT is input by using the training data including APTT as the test parameter. Similarly, the server 1 generates the learning model 50B in which ACT is input by using the training data including ACT as the test parameter. In addition, the server 1 generates the learning model 50C in which both APTT and ACT are input by using the training data including both APTT and ACT as the test parameters. The data of the generated learning models 50A, 50B, and 50C is installed in the terminal 2.

At the time of prediction, when acquiring the patient information of the patient to be predicted, the terminal 2 selects one of the learning models 50 according to the type of the test parameter included in the patient information. Then, the terminal 2 inputs the patient information into the selected learning model 50 to output information regarding an influence of a medicine and the like.

In this manner, in the present embodiment, the plurality of learning models 50 are prepared according to the type of the test parameter. As a result, even in a case where different test items (or test parameters) are used depending on the medical institution or the clinical department, the case can be dealt with by a common system.

Note that, although the learning model 50 may predict the dose of the medicine (heparin or the like) to be administered as described above, for example, the terminal 2 may determine the dose of the medicine to be administered (hereinbelow also referred to as “recommended dose”) on the basis of a predicted value of the test parameter (APTT and/or ACT) after a predetermined time output from the learning model 50.

Specifically, the terminal 2 determines a recommended dose or a recommended dose range on the basis of a predicted value of the test parameter after a predetermined time from the start of medication output from the learning model 50 or a difference between the predicted value of the test parameter after a predetermined time from the start of medication output from the learning model 50 and a measured value of the test parameter input into the learning model 50, that is, a change amount. For example, the terminal 2 holds in advance a table (not illustrated) in which the change amount is associated with the recommended dose or the recommended dose range. When acquiring a predicted value of the test parameter after a predetermined time from the start of the medication by inputting the patient information into the learning model 50, the terminal 2 calculates a difference from the measured value, that is, the change amount. Then, the terminal 2 refers to the aforementioned table and determines a recommended dose or a recommended dose range based on the change amount of the test parameter.

Regarding the management criteria of ACT and APTT, at the time of using ECMO, for example, criteria of “the appropriate time is 180 seconds to 220 seconds” for ACT and “the proper value is 1.5 times to 2.5 times the value at a normal time” for APTT are used in some cases. The recommended dose range of heparin or the like may be determined by comparing the management criteria with the predicted values of ACT and APTT. Note that the management criteria used in the present invention are not limited to those described above.

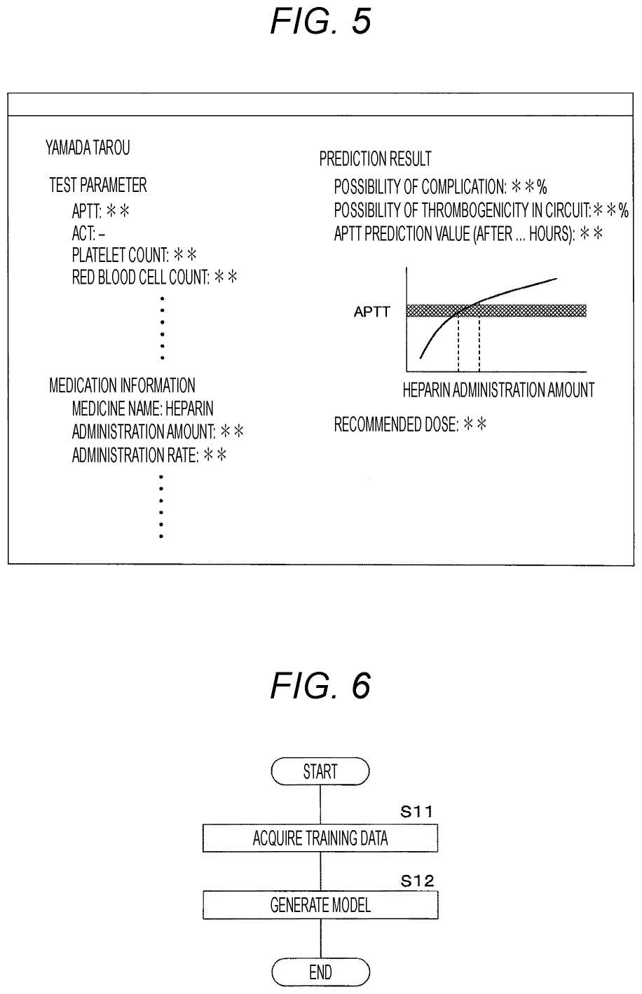

FIG. 5 is an explanatory diagram illustrating a display example of a prediction result. FIG. 5 illustrates a display screen example of a prediction result displayed on the terminal 2. For example, as illustrated in FIG. 5, the terminal 2 displays a list of patient information (test parameters, medication information, patient basic information, and the like) input into the learning model 50 on the left side of the screen. In addition, the terminal 2 displays a list of prediction values (possibility of complication, possibility of thrombogenicity in circuit, value of test parameter (APTT in FIG. 5) after predetermined time from start of medication, and the like) output from the learning model 50 on the right side of the screen. In addition, the terminal 2 displays a graph illustrating the relationship between a dose of a medicine (heparin in FIG. 5) and APTT, and displays a recommended dose of the medicine determined above in association with the graph.

FIG. 6 is a flowchart illustrating processing for generating the learning model 50. With reference to FIG. 6, a description will be given below of some steps of generating the learning model 50 through machine learning.

The control unit 11 of the server 1 acquires a plurality of pieces of training data which are for generating the learning model 50 and differ according to the test parameter (step S11). The training data is data in which a correct output value is associated with patient information including a test parameter of a patient and administration information of a medicine. In the present embodiment, in order to generate a plurality of learning models 50A, 50B, and 50C according to the test parameter, the control unit 11 acquires a plurality of pieces of training data having different types of test parameters.

The control unit 11 generates and optimizes a plurality of learning models 50 (50A, 50B, and 50C) using a plurality of pieces of training data having different types of test parameters, so as to output information regarding an influence of a medicine to be administered to a patient and/or a dose of the medicine to be administered in a case where patient information is input (step S12). Specifically, as described above, the control unit 11 generates the learning model 50 such as a neural network. The control unit 11 generates the learning model 50 by outputting an output value by inputting patient information for training into the learning model 50 and optimizing parameters such as weights between neurons such that the output value approximates a correct value. The control unit 11 uses training data pieces corresponding to respective test parameters to generate the learning models 50A, 50B, and 50C corresponding to the respective test parameters. The control unit 11 then concludes the series of processes.

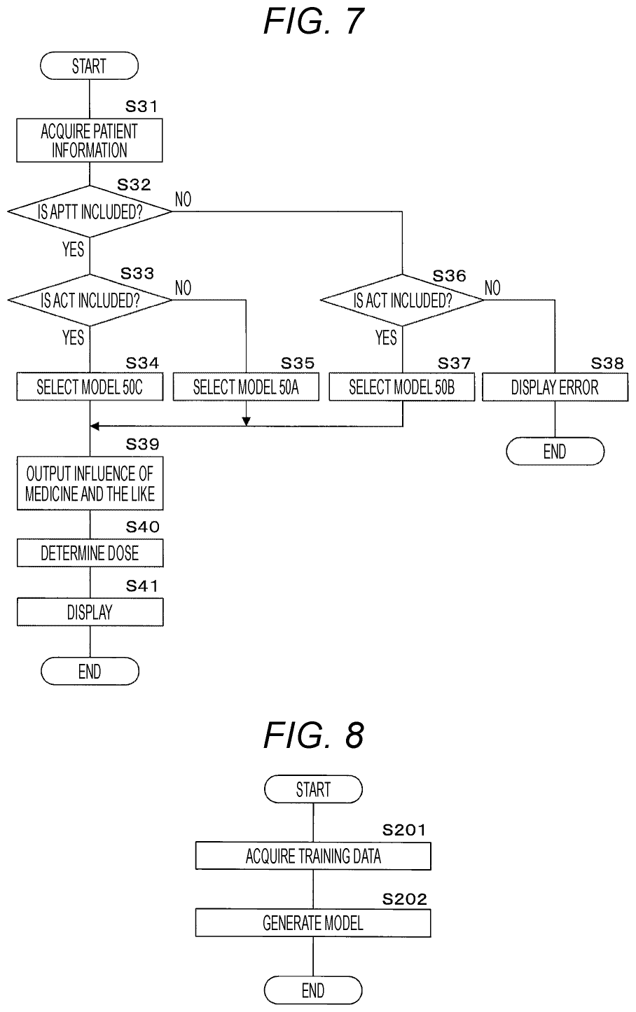

FIG. 7 is a flowchart illustrating prediction processing using the learning model 50. With reference to FIG. 7, a description will be given below of some steps of prediction processing using the learning model 50.

The control unit 21 of the terminal 2 acquires patient information including a test parameter of a patient (step S31). The control unit 21 determines whether APTT is included in the patient information (step S32). In a case where the control unit 21 determines that APTT is included (S32: YES), the control unit 21 determines whether ACT is included in the patient information (step S33). In a case where the control unit 21 determines that ACT is included (S33: YES), the control unit 21 selects the learning model 50C as the learning model 50 to be used for prediction (step S34). In a case where the control unit 21 determines that ACT is not included (S33: NO), the control unit 21 selects the learning model 50A (step S35).

In a case where the control unit 21 determines that APTT is not included in the patient information (S32: NO), the control unit 21 determines whether ACT is included in the patient information (step S36). In a case where the control unit 21 determines that ACT is included (S36: YES), the control unit 21 selects the learning model 50B as the learning model 50 to be used for prediction (step S37). In a case where the control unit 21 determines that ACT is not included (S36: NO), the control unit 21 displays an error message such as “APTT or ACT information is not input.” (step S38), and ends the processing.

The control unit 21 inputs the patient information into the selected learning model 50 to output information regarding an influence of a medicine and/or a dose of the medicine to be administered (step S39). For example, the control unit 21 outputs a condition of a patient (possibility of complication and the like), a state of a device connected to the patient, a test parameter after a predetermined time, and the like as the influence of the medicine. The control unit 21 determines a recommended dose of the medicine on the basis of the test parameter after a predetermined time output from the learning model 50 (step S40). The control unit 21 displays a prediction result including the recommended dose or a recommended dose range of the medicine (step S41), and concludes the series of processes. The administration mechanism of the device for administering the medicine may be automatically controlled on the basis of the recommended dose of the medicine.

As described above, according to the first embodiment, regardless of the type of the test performed on the patient, the influence of the administration of the medicine on the patient or the preferred dose of the medicine can be suitably predicted without being unable to be output due to the data loss.

The information of the test parameter obtained a certain time period, for example, eight hours or more, before the scheduled medication start time is less related to the predicted value of the test parameter after a predetermined time, and in a case where the test parameter after the predetermined time is predicted using such a test parameter, the predicted value may be inaccurate. Therefore, the information of the test performed on the patient a certain time or more before the administration start time may not be used for selection of the learning model and prediction of the test parameter. In other words, selection of the learning model and prediction of the test parameter after a predetermined time may be performed using the parameter tested within a certain time from the scheduled administration start time of the medicine. By doing so, it is possible to obtain a predicted value of the test parameter with higher credibility.

In addition, as for the input of the patient information into the learning model 50, the operator may input the patient information into a prediction program at the time of prediction, or the prediction program may automatically acquire the test parameter and the like already input by the operator at the time of test to select the learning model and predict the test parameter after a predetermined time. Even in a case where the prediction program automatically acquires the patient information already input into the system, an appropriate learning model is selected according to the type of the test performed on the patient to predict the test parameter after a predetermined time. Therefore, it is possible to predict the influence of the administration of the medicine on the patient or the preferable dose of the medicine without the result being unable to be output due to the data loss.

In the first embodiment, a mode for a heparin administration case has been described, but the present embodiment is not limited thereto. For example, the present system may be a system for an acute heart failure case.

### Modification Examples

Specifically, biological information such as urine volume and blood pressure, and a blood test value such as creatinine, which are tested a certain time before the medication, are used as test (parameters. By using predetermined training data, the server 1 generates, according to the types of the test parameters, a plurality of learning models 50 that output information regarding an influence of a medicine such as a diuretic, a cardiotonic, and a vasodilator and/or a dose of the medicine to be administered in a case where patient information including these test parameters and administration information (dose and the like) of the medicine is input. The terminal 2 selects one of the learning models 50 according to the type of the test parameter included in the acquired patient information, and outputs information regarding the influence of the medicine or the dose of the medicine by inputting the patient information.

In this manner, the present system 100 can also be applied to cases other than the heparin administration case.

### Second Embodiment

In the present embodiment, a mode of predicting another type of test parameter (for example, ACT) on the basis of a value of a certain type of test parameter (for example, APTT) will be described. Note that the components identical to those in the foregoing first embodiment are given the same signs and will not be described below.

First, an outline of the present embodiment will be described. In the present embodiment, similarly to the first embodiment, the server 1 generates a learning model 50 that outputs a test parameter after a predetermined time when patient information is input.

Here, the server 1 according to the present embodiment generates the learning model 50 that outputs a second test parameter of a type different from a test parameter included in patient information, that is, a first test parameter to be input. For example, the server 1 generates the learning model 50 that outputs ACT when APTT is input and outputs APTT when ACT is input.

The server 1 generates the learning model 50 using training data in which the second test parameter (ACT or APTT), which is a correct test parameter after a predetermined time, is associated with patient information including the first test parameter (APTT or ACT). That is, the server 1 generates the learning model 50 using training data in which a correct value of ACT is associated with patient information including APTT as the test parameter or a correct value of APTT is associated with patient information including ACT as the test parameter.

The server 1 uses the training data to optimize the learning model 50 that outputs the second test parameter (ACT or APTT) after a predetermined time when patient information including the first test parameter (APTT or ACT) is input. That is, the server 1 generates the learning model 50 that outputs ACT when APTT is input and outputs APTT when ACT is input. For example, the server 1 separately generates a learning model 50A for ACT prediction and a learning model 50B for APTT prediction.

When acquiring patient information, the terminal 2 selects one of the learning models 50 according to the type of the test parameter included in the patient information. Then, by inputting the patient information into the selected learning model 50, the terminal 2 outputs the second test parameter, which is a test parameter after a predetermined time and is of a different type from the first test parameter included in the patient information (that is, the test parameter input into the learning model 50).

In this manner, according to the present embodiment, it is also possible to predict the test parameter (ACT or APTT) different from the parameter (APTT or ACT) tested in advance.

FIG. 8 is a flowchart illustrating processing for generating the learning model 50 according to the second embodiment.

The control unit 11 of the server 1 acquires training data for use in generating the learning model 50 (step S201). The training data is data in which the second test parameter (ACT or APTT), which is a correct test parameter after a predetermined time, is associated with patient information including the first test parameter (APTT or ACT).

Based on the training data, the control unit 11 generates the learning model 50 that outputs the second test parameter after a predetermined time when patient information including the first test parameter is input (step S202). The learning model 50 is, for example, a neural network. The control unit 11 generates different learning models 50 according to the type (APTT or ACT) of the input test parameter. The control unit 11 then concludes the series of processes.

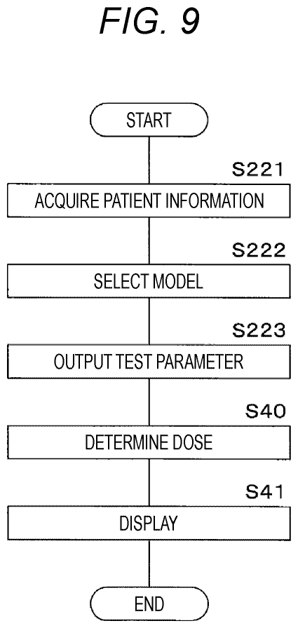

FIG. 9 is a flowchart illustrating prediction processing using the learning model 50 according to the second embodiment.

The control unit 21 of the terminal 2 acquires patient information including a test parameter (step S221). The control unit 21 selects one of the plurality (two types in the present embodiment) of learning models 50A and 50B according to the type of the test parameter included in the patient information (step S222). By inputting the patient information into the selected learning model 50, the control unit 21 outputs the second test parameter, which is a test parameter after a predetermined time and is of a different type from the first test parameter included in the patient information (step S223). The control unit 21 then makes this processing proceed to step S40.

As described above, according to the second embodiment, it is also possible to predict the second test parameter different from the first test parameter tested in advance.

### Third Embodiment

In the present embodiment, separate learning models 50 are prepared according to the elapsed time from test of a patient in order to obtain a test parameter to administration of a medicine.

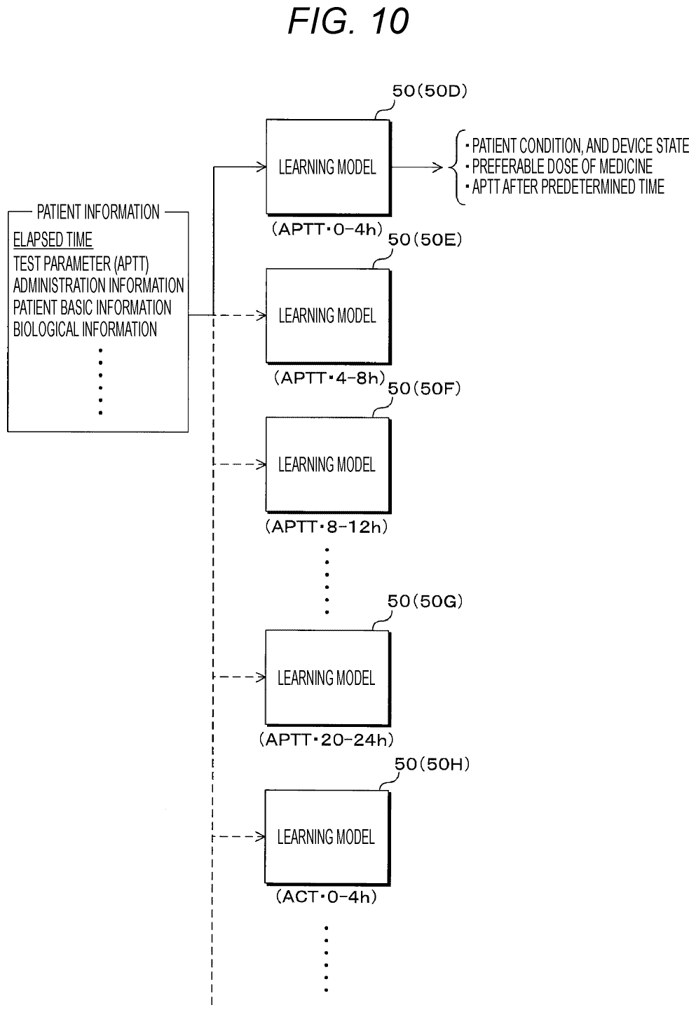

FIG. 10 is an explanatory diagram illustrating an outline of the third embodiment. FIG. 10 illustrates a state in which a plurality of learning models 50D, 50E, 50F, . . . are prepared according to the elapsed time from test of a patient in order to obtain a test parameter to administration of a medicine, and one of the learning models is selected according to the elapsed time included in patient information. With reference to FIG. 10, an outline of the present embodiment will be described below.

Patient information according to the present embodiment includes an elapsed time from test of a patient in order to obtain a test parameter to administration of a medicine. In the present embodiment, by using the elapsed time as one of the explanatory variables, an influence of a medicine administered to a patient and/or a dose of the medicine to be administered are/is accurately predicted.

Specifically, the server 1 groups the elapsed times every predetermined time period and prepares a plurality of different learning models 50D, 50E, 50F, . . . every predetermined time period. FIG. 10 illustrates that the learning models 50 are prepared every 4 hours, that is, the learning model 50D of which the elapsed time is 0 to 4 hours, the learning model 50E of which the elapsed time is 4 to 8 hours, the learning model 50F of which the elapsed time is 8 to 12 hours, . . . the learning model 50G of which the elapsed time is 20 to 24 hours are prepared. The server 1 groups pieces of the training data according to the type of the test parameter and the elapsed time, and generates the different learning models 50D, 50E, 50F, . . . according to the type of the test parameter and the elapsed time on the basis of the training data of the respective groups.

When acquiring patient information, the terminal 2 selects one of the learning models 50 according to the type of the test parameter included in the patient information and the elapsed time. Then, the terminal 2 receives as input the patient information into the selected learning model 50 to output information regarding an influence of a medicine (condition of patient, state of connected device, test parameter after predetermined time, and the like) and/or a dose of the medicine to be administered.

As described above, according to the present embodiment, by using the elapsed time from test of a patient to administration of a medicine as the explanatory variable, the influence of the medicine and the like can be suitably predicted. In particular, in the present embodiment, the prediction accuracy can be enhanced by preparing the plurality of learning models 50D, 50E, 50F . . . according to the elapsed time.

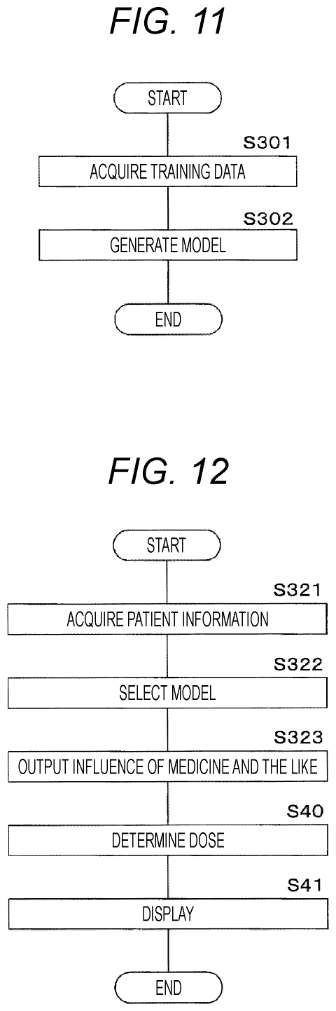

FIG. 11 is a flowchart illustrating processing for generating the learning model 50 according to the third embodiment.

The control unit 11 of the server 1 acquires pieces of training data which are for generating the learning models 50 and differ according to the type of the test parameter and the elapsed time from test of a patient to administration of a medicine (step S301). Specifically, the control unit 11 acquires pieces of training data of which the elapsed times are different every predetermined time period (for example, 4 hours).

Based on the training data, the control unit 11 generates, according to the type of the test parameter and the elapsed time, the plurality of learning models 50 that output information regarding an influence of a medicine or a dose of the medicine to be administered in a case where patient information including the test parameter and the elapsed time is input (step S302). That is, the control unit 11 generates the plurality of learning models 50D, 50E, 50F . . . which have different types of test parameters and of which the elapsed times are different every predetermined time period. The control unit 11 then concludes the series of processes.

FIG. 12 is a flowchart illustrating prediction processing using the learning model 50 according to the third embodiment.

The control unit 21 of the terminal 2 acquires patient information including a test parameter and an elapsed time (step S321). The control unit 21 selects one of the plurality of learning models 50D, 50E, 50F . . . according to the type of the test parameter included in the patient information and the elapsed time (step S322). The control unit 21 inputs the patient information into the selected learning model 50 to output information regarding an influence of a medicine or a dose of the medicine to be administered (step S323). The control unit 21 then makes this processing proceed to step S40.

As described above, according to the third embodiment, by using the elapsed time from test of a patient to administration of a medicine as the explanatory variable, the influence of the medicine and the like can be suitably predicted.

It should be understood that the embodiments disclosed herein are illustrative in all respects and are not restrictive. The scope of the present invention should be defined by the claims rather than the above meaning, and is intended to include all conceivable modifications and variations within the meaning and scope equivalent to the claims.

Some or all of the subject matters described in the respective embodiments can be combined together. In addition, some or all of the independent claims and their dependent claims described in the “CLAIMS” can be combined together, regardless of their dependent relationships. Furthermore, a form (multiple dependent claim form) in which a claim dependent on two or more other claims is described is used in the “CLAIMS”; however, the claim form is not limited thereto. The present invention may be described using a form in which a multiple dependent claim is dependent on at least one multiple dependent claim.

What is claimed is:

1. A medication management system for managing medication for a patient, comprising:
a display device;
a memory that stores a program; and
a processor configured to execute the program to:
acquire patient information about the patient, a first test value of a laboratory test that was performed on the patient, and medicine information including a dose of a medicine to be administered to the patient,
execute a call to a machine learning model with the patient information, the first test value, and the medicine information to determine a second test value expected at a predetermined time after the medicine is administered, the machine learning model having been trained with test values obtained before and after administration of the medicine to different patients, and medicine information including a dose of the medicine to be administered to the different patients,
determine a recommended dose or a preferred range of doses for the medicine based on the second test value,
generate data of a graph showing a relationship between doses of the medicine and test values, and
control the display device to display the graph and the recommended dose or the preferred range on the graph.

2. The medication management system according to claim 1, wherein
the processor is configured to execute the program to control the display device to display the second test value together with the graph.

3. The medication management system according to claim 1, wherein
the machine learning model has been trained to output a possibility of complication after administration of the medicine, and
the processor is configured to execute the program to control the display device to further display the possibility of complication.

4. The medication management system according to claim 1, wherein
the processor is configured to execute the program to control the display device to display the patient information together with the graph.

5. The medication management system according to claim 1, wherein
the memory stores a table associating differences in test values before and after administration of the medicine with recommended doses or preferred ranges of doses for the medicine, and
the processor is configured to execute the program to acquire a difference between the first and second test values, and determine the recommended dose or the preferred range based on the difference and the table stored in the memory.

6. The medication management system according to claim 5, wherein
the processor is configured to execute the program to control the display device to display the preferred range together with the graph.

7. A method for managing medication for a patient, comprising:
acquiring patient information about the patient, a first test value of a laboratory test that was performed on the patient, and medicine information including a dose of a medicine to be administered to the patient;
executing a call to a machine learning model with the patient information, the first test value, and the medicine information to determine a second test value expected at a predetermined time after the medicine is administered, the machine learning model having been trained with test values obtained before and after administration of the medicine to different patients, and medicine information including a dose of the medicine to be administered to the different patients;
determining a recommended dose or a preferred range of doses for the medicine based on the second test value;
generating data of a graph showing a relationship between doses of the medicine and test values; and
displaying the graph and the recommended dose or the preferred range on the graph.

8. The method according to claim 7, further comprising:
displaying the second test value together with the graph.

9. The method according to claim 7, wherein
the machine learning model has been trained to output a possibility of complication after administration of the medicine, and
the method comprises further displaying the possibility of complication.

10. The method according to claim 7, further comprising:
displaying the patient information together with the graph.

11. The method according to claim 7, further comprising:
storing a table associating differences in test values before and after administration of the medicine with recommended doses or preferred ranges of doses for the medicine, and
determining a difference between the first and second test values, wherein
the recommended dose or the preferred range is determined based on the difference and the table.

12. The method according to claim 11, further comprising:
displaying the preferred range together with the graph.

13. A medication management apparatus for managing medication for a patient, comprising
a memory that stores a program and a plurality of machine learning models, wherein
each machine learning model being configured to receive a different test value of a laboratory test performed on a patient, patient information about the patient, and medicine information including a dose of a medicine to be administered to the patient, and output information indicating effectiveness of the medicine or a dose of the medicine,
each machine learning model having been trained with different test values obtained before and after administration of the medicine to different patients; and
a processor configured to execute the program to:
acquire patient information about a patient, at least a first test value for the patient, and medicine information indicating a medicine to be administered to the patient,
select one of the machine learning models that can accept an input of the first test value,
execute a call to said one of the machine learning model with the patient information, the first test value, and the medicine information to generate information indicating effectiveness of the medicine or a dose of the medicine, and
output the generated information.

14. The medication management apparatus according to claim 13, wherein
the first test value is acquired through a laboratory test that was performed within a predetermined time from a scheduled administration start time of the medicine.

15. The medication management apparatus according to claim 13, wherein
the information that is output from each of the machine learning models indicates a condition of the patient.

16. The medication management apparatus according to claim 13, wherein
the information that is output from said one of the machine learning models indicates a second test value expected at a predetermined time after the medicine is administered, and
the processor is configured to execute the program to determine a recommended dose of the medicine based on the second test value.

17. The medication management apparatus according to claim 13, wherein
the medicine is heparin, and
said at least a first test value is an activated partial thromboplastin time (APTT), an activated clotting time (ACT), or a combination thereof.

18. The medication management apparatus according to claim 13, wherein
each machine learning model further receives, as input, a time from the laboratory test to administration of the medicine.

19. The medication management apparatus according to claim 13, wherein
the processor is configured to execute the program to determine whether any one of the machine learning models can receive the first test value as input.

20. The medication management apparatus according to claim 13, further comprising:
an interface circuit connectable to a display device, wherein
the processor is configured to execute the program to output the generated information to the display device and cause the display device to display the generated information together with the patient information and the medicine information.
